(19) United States
(12) Patent Application Publication
(10) Pub. No.: US 2009/0311732 A1
(43) Pub. Date: Dec. 17, 2009

(54) ANALYTICAL METHOD FOR ANALYZING C-TERMINUS TRUNCATION

(75) Inventors: Mara Rossi, Rome (IT); Irene Cecchini, Rome (IT)

Correspondence Address: SALIWANCHIK LLOYD & SALIWANCHIK;A PROFESSIONAL ASSOCIATION, PO Box 142950, GAINESVILLE, FL, 32614, US

(73) Assignee: Ares Trading S.A., Aubonne (CH)

(21) Appl. No.: 12/520,196

(22) PCT Filed: Dec. 20, 2007
(86) PCT No.: PCT/EP2007/064351

§ 371 Date: Jun. 19, 2009

Related U.S. Application Data
(60) Provisional application No. 60/886,382, filed on Jan. 24, 2007.

(30) Foreign Application Priority Data
Dec. 22, 2006 (EP) 06026811.7

(51) Int. Cl. C12Q 1/37

(52) U.S. Cl. 435/23

(57) ABSTRACT

This invention relates to analytical methods for quantification of truncation at the C-terminus of an Fc-containing protein.

### FIELD OF THE INVENTION

This invention provides analytical methods for quantification of truncation at the C-terminus of an Fc-containing protein such as e.g. antibodies and Fc-fusion proteins. More specifically, the methods of the present invention allow testing the proportion of said protein for which the C-terminal lysine has been cleaved off by an endoprotease present within the producer cell.

### BACKGROUND OF THE INVENTION

Antibodies and Fc-fusion proteins are useful as therapeutic proteins for the treatment of a number of diseases. While the non-processed sequences of such Fc-containing proteins are known, in practice the produced proteins are heterogeneous. One of the commonly observed modifications resides in the C-terminus of the Fc chain of the protein, which is caused by the activity of basic carboxypeptidases inside the cell from which the protein is produced. These enzymes remove the C-terminal lysine residue from a fraction of the produced proteins, thereby generating two variants of the produced protein. These variants are usually referred to as Lysine variants, namely Lys0 (C-terminal lysine cleaved-off) and Lys1 (C-terminal lysine present).

The characterization, development, manufacture, and release of an antibody or Fc-fusion protein requires an analytical tool for quantifying the percentage of Lys0 and Lys1 variants respectively, in particular if the therapeutic protein is prepared for human administration. For example, such a tool may be used for analyzing different development lots, for characterizing the protein in the frame of a marketing authorization submission, or for lot release testing.

Some methods for assessing C-terminal lysine truncation of antibodies have been described in the art.

Santora et al. (1999) discloses a method for analyzing C-termini Lys variants of a recombinant, human anti-tumor necrosis factor monoclonal antibody. Different C-termini Lys variants were separated and collected from a cation-exchange liquid chromatography column and subsequently analyzed by capillary isoelectric focusing methods and mass spectrometry.

Lazar et al. (2004) teaches a method for analyzing C-terminal lysine distribution of monoclonal antibodies using matrix-assisted laser desorption/ionization mass spectrometry.

Dillon et al. (2004) discloses an analytical reversed-phase high-performance liquid chromatography-electrospray ionization mass spectrometry method for characterization of recombinant antibodies, said method allowing the separation of lysine variants from intact IgG1 antibodies.

The analytical methods for assessing C-terminal lysine truncation of antibodies that are available in the art thus involve an analysis by mass spectrometry or by cation exchange chromatography. Such methods strictly depend on the pI range and/or on the charge heterogeneity of the molecules. In other terms, the methodology itself can be used for analyzing any antibody, but the scientist must set up new analytical conditions for each specific antibody being analyzed. These conditions depend on the pI range and/or on the charge heterogeneity of the specific antibody being analyzed.

Therefore, there is a need for a quantitative analytical method allowing the assessment of C-terminal lysine truncation of Fc-containing proteins that can be applied to any antibody or Fc-fusion protein without the requirement of setting up new analytical conditions for each specific protein being analyzed.

### SUMMARY OF THE INVENTION

The present invention stems from the finding of a method for quantification of truncation at the C-terminus of any protein comprising SEQ ID NO: 1 at its C-terminal extremity such as, e.g., antibodies and Fc-fusion proteins. This method, which is based on hydrolyzing the protein to be analyzed by a Lys-C endoproteinase, is particularly advantageous because it does not depend on the pI range or the isoform profile of the protein to be analyzed. It can thus be applied to any protein comprising SEQ ID NO: 1 at its C-terminal extremity without the need of developing specific sample treatments or specific analytical conditions on a case by case basis.

Therefore, one embodiment of the invention is a method for measuring, determining and/or estimating the relative amount of a first protein and of a second protein in a sample, said method comprising the steps of:
a) providing a sample comprising said proteins;
b) hydrolyzing said proteins by a Lys-C endoproteinase; and
c) separating the hydrolysate obtained in step (b) by a method capable of distinguishing between peptides having a difference of one amino acid in length;
   wherein:
(i) the non-processed sequence of said first protein is identical to the non-processed sequence of said second protein;
(ii) said first protein comprises a peptide of Formula I at its C-terminal extremity:

Lys-(Xaa)z-Lys Formula I
(iii) said second protein comprises a peptide of Formula II at its C-terminal extremity:

Lys-(Xaa)z Formula II
(iv) Xaa is any amino acid except of Lys; and
(v) 5≦z≦20.

In another embodiment of the invention, manufacturing lots of a therapeutic protein are validated according to the method disclosed herein.

In a third embodiment of the invention, a peptide of Formula III ((Xaa)z-Lys) or of Formula IV ((Xaa)z) is used for the detection of intact Fc-containing proteins or of truncated Fc-containing proteins, respectively.

### BRIEF DESCRIPTION OF THE FIGURES

FIG. 1 is a schematic representation of an embodiment of the method in accordance with the invention. A proteolysis with a Lys-C endoproteinase is first carried out on a sample comprising a protein comprising an Fc chain. Then, a Reverse Phase High Performance Liquid Chromatography (RP-HPLC) capable of detecting and quantifying the truncation at the C-terminus is carried out. On FIG. 1, the fragments having C-termini with lysine cleaved off (proteolysis of a Lys0 variant of the protein) represent the main part of the eluted fragments, and they are well separated from the fragments carrying the lysine (proteolysis of a Lys1 variant of the protein). The Fc chain depicted in FIG. 1 corresponds to SEQ ID NO: 4 of the Sequence Listing. The intact fragment corresponds to SEQ ID NO: 2. The truncated fragment corresponds to SEQ ID NO: 3.

FIG. 2 depicts a typical RP-HPLC analytical profile obtained when carrying out Examples 1 to 3. This profile was obtained for an Fc-fusion protein comprising a fragment of the TACI receptor as described in WO 02/094852.

FIG. 3 depicts the RP-HPLC analytical profiles when performing the method described in Examples 1 to 3 on six different proteins comprising the sequence of SEQ ID NO: 1 at their C-terminal extremity. “TACI-Fc” stands for an Fc-fusion protein comprising a fragment of the transmembrane activator and calcium-modulator and cyclophilin ligand interactor (TACI) receptor as described in WO 02/094852. “Anti-CD4 Mab” is the anti-CD4 antibody 6G5 described in WO 97/13852. “Anti-CD25 Mab” is the anti-CD25 antibody AB12 as described in WO 2004/045512. “IFN-Fc No. 1” is an Fc-fusion protein comprising a fragment of IFN-beta as described in WO 2005/001025. “IFN-Fc No. 2” is a second Fc-fusion protein comprising a fragment of IFN-beta. “Anti-CD11a Mab” is the anti-CD11a antibody F(ab)-8 as described WO 98/23761.

### BRIEF DESCRIPTION OF THE SEQUENCE LISTING

SEQ ID NO: 1 corresponds to nine C-terminal amino acids that are usually present in heavy chains of antibodies and in Fc chains.

SEQ ID NO: 2 corresponds to the intact sequence present in heavy chains of antibodies and in Fc chains (Lys1 variant)

SEQ ID NO: 3 corresponds to the truncated sequence present in heavy chains of antibodies and in Fc chains (Lys0 variant).

SEQ ID NO: 4 corresponds to the sequence of an exemplary Fc region comprising SEQ ID NO: 1 at its C-terminal extremity.

### DETAILED DESCRIPTION OF THE INVENTION

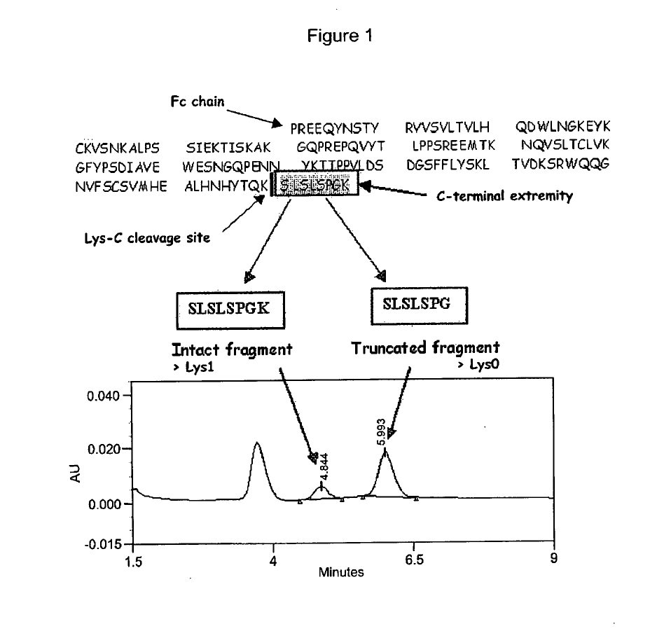

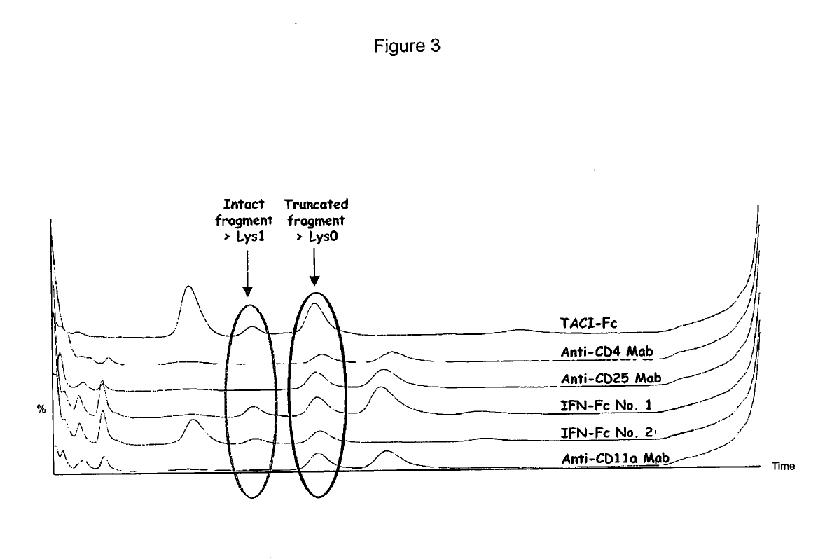

The non-processed sequence of heavy chains of antibodies and of Fc chains comprises SEQ ID NO: 1 at their C-terminal extremity. The C-terminus of heavy chains of antibodies and of Fc chains may be proteolitycally processed by carboxypeptidases within the cell, thus generating heavy chains and Fc chains lacking the C-terminal lysine. The present invention stems from the finding that C-terminal truncation of heavy chains of antibodies and/or of Fc chains can be determined by a method in which a Lys-C endoproteinase is used. Upon hydrolysis of heavy chains or Fc chains by the Lys-C endoproteinase, a fragment of SEQ ID NO: 2 is generated when said chains have not been processed by any carboxypeptidase within the cell, while a fragment of SEQ ID NO: 3 is generated when said chains have been processed by a carboxypeptidase within the cell. Such fragments of SEQ ID Nos. 2 and 3 may then be separated by chromatography, thus allowing the percentage of proteins for which the C-terminal lysine has been cleaved off (see FIG. 1) to be estimated. In addition, it has surprisingly been found that this method is applicable for the analysis of any Fc-containing protein without the need of changing the experimental conditions (see FIG. 3).

Therefore, one embodiment of the invention is a method for measuring, determining and/or estimating the relative amount of a first protein and of a second protein in a sample, said method comprising the steps of:
a) providing a sample comprising said proteins;
b) hydrolyzing said proteins by a Lys-C endoproteinase; and
c) separating the hydrolysate obtained in step (b) by a method capable of distinguishing between peptides having a difference of one amino acid in length;
wherein:
   (i) the non-processed sequence of said first protein is identical to the non-processed sequence of said second protein;
   (ii) said first protein comprises a peptide of Formula I at its C-terminal extremity:

Lys-(Xaa)z-Lys Formula I
(iii) said second protein comprises a peptide of Formula II at its C-terminal extremity:

Lys-(Xaa)z Formula II
(iv) Xaa is any amino acid except of Lys; and
(v) 5≦z≦20.

As used herein, the term “non-processed sequence” of a protein refers to the amino acid sequence as encoded by the corresponding messenger RNA, before any proteolytic processing of the protein has taken place within the cell in which the protein is expressed. In one specific embodiment, the second protein corresponds to a first protein in which the C-terminal lysine has been cleaved off. In the frame of the present application, the feature “the non-processed sequence of said first protein is identical to the non-processed sequence of said second protein” may thus alternatively be defined as follows: “the sequence of said first protein is identical to the sequence of said second protein except for the additional presence of a C-terminal Lysine in said first protein”.

As used herein, the term “protein according to the invention” refers both to the first and to the second protein.

The relative amount of the first protein and of the second protein may for example be expressed as a percentage or as a ratio. When expressed as a percentage, 100% corresponds to the total amount of proteins according to the invention, i.e., the amount of the first protein and of the second protein.

In the method according to the invention, z may have any value comprised within a range of 3 to 50, 3 to 40, 3 to 30, 3 to 20, 3 to 15, 3 to 10, 3 to 9, 3 to 8, 3 to 7, 3 to 6, 4 to 50, 4 to 40, 4 to 30, 4 to 20, 4 to 15, 4 to 10, 4 to 9, 4 to 8, 4 to 7, 4 to 6, 5 to 50, 5 to 40, 5 to 30, 5 to 20, 5 to 15, 5 to 10, 5 to 9, 5 to 8, 5 to 7, or 5 to 6. In a specific embodiment, z has a value of 7.

Although each of these terms has a distinct meaning, the terms “comprising” and “consisting of” may be interchanged for one another throughout the instant application. The term “having” has the same meaning as the term “comprising”.

As used herein, the term “Lys-C endoproteinase” is synonymous with the term “Lysyl endopeptidase” and refers to an enzyme that cleaves the bond between a lysine and any amino acid within a polypeptide and/or a protein (see ENZYME/UniProtKB/Swiss-Prot Accession No. EC 3.4.21.50). Such enzymes include, but are not limited to, endoproteinases endoproteinases recombinantly produced using a coding sequence cloned from Lysobacter enzymogenes (UniProtKB/Swiss-Prot Accession No. Q7M135), Pseudomonas aeruginosa (Ps-1; UniProtKB/Swiss-Prot Accession No. Q9HWK6) and Achromobacter lyticus (UniProtKB/Swiss-Prot Accession No. P15636). In one specific embodiment of the invention, the Lys-C endoproteinase corresponds to the Lysobacter enzymogenes endoproteinase. In another embodiment, the Lys-C endoproteinase is purified from L. enzymogenes, P. aeruginosa or A. lyticus.

In one embodiment, the protein according to the invention is an Fc-containing protein such as, e.g., an antibody or an Fc-fusion protein. In specific embodiments, the Fc-containing proteins are chimeric proteins consisting of the effector region of a protein, such as e.g. the Fab region of an antibody or the binding region of a receptor, fused to the Fc region of an immunoglobulin including, but not limited to immunoglobulin G (IgG).

The term “Fc-containing protein”, as used herein, is meant to encompass proteins, in particular therapeutic proteins, comprising an immunoglobulin-derived moiety, which will be called herein the “Fc-moiety”, and another moiety, either derived from the same or from a different protein than the Fc-moiety, which will be called herein the “therapeutic moiety”, irrespective of whether or not treatment of disease is intended. The Fc-moiety may for example have the sequence of SEQ ID NO: 4. Other Fc-moieties may have an amino acid sequence which is at least 99%, 98%, 95%, 90%, 85% or 80% identical to the sequence according to SEQ ID NO: 4. The recombinant polypeptide fused to the Fc-moiety may correspond to any polypeptide of interest, in particular for polypeptides for which cellular secretion and/or production in a cell is desired. As used herein, the term Fc-containing protein encompasses both antibodies and Fc-fusion proteins.

As used herein, the term “antibody” refers to an Fc-containing protein wherein the therapeutic moiety comprises at least one variable domain of an immunoglobulin (Ig). Preferred immunoglobulins are mammalian immunoglobulins. More preferred immunoglobulins are camelid immunoglobulins. Even more preferred immunoglobulins are rodent immunoglobulins, in particular from rat or mouse. Most preferred immunoglobulins are primate immunoglobulins, in particular human immunoglobulins.

The term “Fc-fusion protein” refers to an Fc-containing protein wherein the therapeutic moiety is a protein other than a variable domain of an immunoglobulin such as, e.g., the extracellular domain of a receptor or a domain of a soluble protein.

The Fc-moiety may be derived from a human or animal immunoglobulin (Ig) that is preferably an IgG. The IgG may be an IgG₁, IgG₂, IgG₃ or IgG₄. The Fc-moiety may comprise all or a part of the constant region domains of an immunoglobulin. It is preferred that the Fc-moiety comprises at least a CH₂ and CH₃ domain. It is further preferred that the Fc-moiety comprises the Ig hinge region, the CH₂ and the CH₃ domain. Particularly preferred the Fc-moiety comprises the IgG CH₂ and the CH₃ domain, with or without the hinge region.

The Fc-containing protein of the invention may be a monomer or dimer. The Fc-containing protein may also be a “pseudo-dimer”, containing a dimeric Fc-moiety (e.g. a dimer of two disulfide-bridged hinge-CH2-CH3 constructs), of which only one is fused to a therapeutic moiety. The Fc-containing protein may be a heterodimer, containing two different therapeutic moieties, or a homodimer, containing two copies of a single therapeutic moiety. Preferably, the Fc-fusion protein is a dimer. It is also preferred that the Fc-containing protein of the invention is a homodimer.

In accordance with the present invention, the Fc-moiety may also be modified in order to modulate effector functions. For instance, the following Fc mutations, according to EU index positions (Kabat et al., 1991), can be introduced if the Fc-moiety is derived from IgG₁:
T250Q/M428L
M252Y/S254T/T256E+H433K/N434F
E233P/L234V/L235A/ΔG236+A327G/A330S/P331S
E333A; K322A.

Further Fc mutations may e.g. be the substitutions at EU index positions selected from 330, 331 234, or 235, or combinations thereof. An amino acid substitution at EU index position 297 located in the CH2 domain may also be introduced into the Fc-moiety in the context of the present invention, eliminating a potential site of N-linked carbohydrate attachment. The cysteine residue at EU index position 220 may also be replaced with a serine residue, eliminating the cysteine residue that normally forms disulfide bonds with the immunoglobulin light chain constant region.

The therapeutic moiety of the Fc-containing protein may e.g. be or be derived from EPO, TPO, Growth Hormone, Interferon-alpha, Interferon-beta, Interferon-gamma, PDGF-beta, VEGF, IL-1alpha, IL-1beta, IL-2, IL-4, IL-5, IL-8, IL-10, IL-12, IL-18, IL-18 binding protein, TGF-beta, TNF-alpha, or TNF-beta.

The therapeutic moiety the Fc-containing protein may also be derived from a receptor, e.g a transmembrane receptor, preferably be or be derived from the extracellular domain of a receptor, and in particular a ligand binding fragment of the extracellular part or domain of a given receptor. Examples for therapeutically interesting receptors are CD2, CD3, CD4, CD8, CD11a, CD14, CD18, CD20, CD22, CD23, CD25, CD33, CD40, CD44, CD52, CD74, CD80, CD86, CD147, CD164, IL-2 receptor, IL4 receptor, IL-6 receptor, IL-12 receptor, IL-18 receptor subunits (IL-18R-alpha, IL-18R-beta), EGF receptor, MIF receptor, VEGF receptor, integrin alpha 4 10 beta 7, the integrin VLA4, B2 integrins, TRAIL receptors 1, 2, 3, and 4, RANK, RANK ligand, epithelial cell adhesion molecule (EpCAM), intercellular adhesion molecule-3 (ICAM-3), CTLA4 (which is a cytotoxic T lymphocyte-associated antigen), Fc-gamma-I receptor, HLA-DR 10 beta, HLA-DR antigen, L-selectin, a fragment og a receptor belonging to the TNFR superfamily such as, e.g., a fragment derived from the extracellular domain of TNFR1 (p55), TNFR2 (p75), OX40, Osteoprotegerin, CD27, CD30, CD40, RANK, DR3, Fas ligand, TRAIL-R1, TRAIL-R2, TRAIL-R3, TAIL-R4, NGFR, AITR, BAFFR, BCMA or TACI.

One embodiment of the invention is a method wherein step (c) of the method distinguishes between peptides of Formula III and Formula IV:

(Xaa)z-Lys Formula III

(Xaa)z Formula IV

Another embodiment of the invention is a method wherein the non-processed sequence of the protein comprises a polymorphic variant of SEQ ID NO: 1 at its C-terminal extremity provided that said polymorphic variant of SEQ ID NO: 1 falls within the scope of Formula I.

As used herein, the term “polymorphic variant” of a given sequence refers to a sequence in which one or more amino acids have been substituted by a different amino acid as compared to said given sequence. In a specific embodiment, said substitution is a conservative substitution as indicated in Tables 1 to 3 herebelow. In another specific embodiment, said polymorphic variant comprises less than 7, 6, 5, 4, 3 or 2 polymorphic variations compared to said given sequence. In another specific embodiment, said polymorphic variant is a single polymorphic variant. In another specific embodiment, said polymorphic variant is at least 80%, 85%, 90%, 95%, 96%, 97%, 98% or 99% identical to said given sequence.

TABLE 1
First Group of Synonymous Amino Acids

| | |
|---|---|
| Amino Acid | Synonymous Group |

| | |
|---|---|
| Ser | Ser, Thr, Gly, Asn |
| Arg | Arg, Gln, Glu, His |
| Leu | Ile, Phe, Tyr, Met, Val, Leu |
| Pro | Gly, Ala, Thr, Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Ala, Thr, Pro, Ser, Gly |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Ser, Thr, Cys |
| His | Glu, Gln, Thr, Arg, His |
| Gln | Glu, Asn, His, Thr, Arg, Gln |
| Asn | Gln, Asp, Ser, Asn |
| Asp | Glu, Asn, Asp |
| Glu | Asp, Asn, Gln, His, Arg, Glu |
| Met | Phe, Ile, Val, Leu, Met |
| Trp | Trp |

TABLE 2
Second Group of Synonymous Amino Acids

| | |
|---|---|
| Amino Acid | Synonymous Group |

| | |
|---|---|
| Ser | Ser |
| Arg | His, Arg |
| Leu | Leu, Ile, Phe, Met |
| Pro | Ala, Pro |
| Thr | Thr |
| Ala | Pro, Ala |
| Val | Val, Met, Ile |
| Gly | Gly |
| Ile | Ile, Met, Phe, Val, Leu |
| Phe | Met, Tyr, Ile, Leu, Phe |
| Tyr | Phe, Tyr |
| Cys | Cys, Ser |
| His | His, Gln, Arg |
| Gln | Glu, Gln, His |
| Asn | Asp, Asn |
| Asp | Asp, Asn |
| Glu | Glu, Gln |
| Met | Met, Phe, Ile, Val, Leu |
| Trp | Trp |

TABLE 3
Third Group of Synonymous Amino Acids

| | |
|---|---|
| Amino Acid | Synonymous Group |

| | |
|---|---|
| Ser | Ser |
| Arg | Arg |
| Leu | Leu, Ile, Met |
| Pro | Pro |
| Thr | Thr |
| Ala | Ala |
| Val | Val |
| Gly | Gly |
| Ile | Ile, Met, Leu |
| Phe | Phe |
| Tyr | Tyr |
| Cys | Cys, Ser |
| His | His |
| Gln | Gln |
| Asn | Asn |
| Asp | Asp |
| Glu | Glu |
| Met | Met, Ile, Leu |
| Trp | Met |

Another embodiment of the invention is a method wherein the non-processed sequence of said protein comprises the sequence of SEQ ID NO: 1 at its C-terminal extremity.

Another embodiment of the invention is a method wherein step (c) of the method distinguishes between peptides having a sequence of SEQ ID NO: 2 and peptides having a sequence of SEQ ID NO: 3.

Step (c) of the method according to the invention may be carried out using any method that allows one to distinguish between peptides having a difference of one amino acid in length. Such methods are well-known in the art and include, e.g., chromatography and mass spectrometry.

One embodiment of the invention is a method wherein step (c) is carried out by chromatography. In one specific embodiment, step (c) is carried out by Reverse Phase High Performance Liquid Chromatography (RP-HPLC).

When step (c) is carried out by RP-HPLC, the following conditions may for example be used. The eluents may for example be the following:
0.10% Trifluoroacetic acid in water (referred to as eluent A)
0.08% Trifluoroacetic acid in Acetonitrile 70% (referred to as eluent B)
   The column temperature may for example be of about +40° C. The run duration may for example be of about 15 minutes. The column flow rate may for example be of about 1 mL/min. The linear gradient may for example be set up as indicated in table 4 of Example 2.

In a specific embodiment, the RP-HPLC according to step (c) of the present invention is carried out as detailed in Example 2. However, numerous methods for carrying out RP-HPLCs are known in the art and the skilled person could routinely set up different conditions for separating peptides of Formula III from peptides of Formula IV.

Another embodiment of the invention is a method wherein the reaction of step (b) is carried out with a range of about 20 to 1, 15 to 2 or 10 to 4 μg of said Lys-C endoproteinase and with a range of about 500 to 25, 400 to 50, 300 to 75 or 200 to 125 μg of said protein. In one specific embodiment, the reaction of step (b) is carried out with (i) a range of about 2 to about 10 μg of said Lys-C endoproteinase; and (ii) about 100 μg of said protein. In another specific embodiment, the reaction of step (b) is carried out with (i) about 2.5 μg or about 5 μg of said Lys-C endoproteinase; and (ii) about 100 μg of said protein.

Another embodiment of the invention is a method wherein the reaction of step (b) is carried out for about 5, 4, 3, 2 or 1 hours. In a specific embodiment, the reaction of step (b) is carried out for about 2 hours.

A further embodiment of the invention is a method wherein the reaction of step (b) is carried out at about 42, 40, 37, 35 or 30° C. In a specific embodiment, the reaction of step (b) is carried out at about 37° C.

One specific embodiment of the invention is a method wherein step (b) is carried out with a buffer at about pH 7.5 comprising 0.5 M Tris-HCl and 2 mM EDTA. Alternatively, the skilled in the art could use other buffers.

A further embodiment of the invention is a method comprising the step of stopping the reaction of step (b) before carrying out step (c). The reaction of step (b) may be stopped by any method known to those of skill in the art. In one specific embodiment, the reaction of step (b) is stopped by adding, e.g., trifluoroacetic acid (TFA). In another specific embodiment, the reaction of step (b) is stopped by adding TFA at a concentration of 10%.

The sample comprising the protein may for example correspond to a purified protein, e.g. when testing development lots, or to a pharmaceutical preparation, e.g. when characterizing a protein in the frame of a marketing authorization submission or when carrying out lot release testing.

In one embodiment of the invention, the protein according to the invention is an antibody. The antibody may be a chimeric antibody, a humanized antibody, a fully humanized antibody or a human antibody. The antibody may either be produced in a host cell transfected with one, two or more polynucleotides coding for the antibody or produced from an hybridoma.

Preferably, said antibody is directed against a protein selected from the group consisting of CD3 (e.g. OKT3, NI-0401), CD11a (e.g. efalizumab), CD4 (e.g. zanolimumab, TNX-355), CD20 (e.g. ibritumomab tiuxetan, rituximab, tositumomab, ocrelizumab, ofatumumab, IMMU-106, TRU-015, AME-133, GA-101), CD23 (e.g. lumiliximab), CD22 (e.g. epratuzumab), CD25 (e.g. basiliximab, daclizumab), the epidermal growth factor receptor (EGFR) (e.g. panitumumab, cetuximab, zalutumumab, MDX-214), CD30 (e.g MDX-060), the cell surface glycoprotein CD52 (e.g. alemtuzumab), CD80 (e.g. galiximab), the platelet GPIIb/IIIa receptor (e.g. abciximab), TNF alpha (e.g. infliximab, adalimumab, golimumab), the interleukin-6 receptor (e.g. tocilizumab,), carcinoembryonic antigen (CEA) (e.g. 99mTc-besilesomab), alpha-4/beta-1 integrin (VLA4) (e.g. natalizumab), alpha-5/beta-1 integrin (VLA5) (e.g. volociximab), VEGF (e.g. bevacizumab, ranibizumab), immunoglobulin E (IgE) (e.g. omalizumab), HER-2/neu (e.g. trastuzumab), the prostate specific membrane antigen (PSMA) (e.g. 111In-capromab pendetide, MDX-070), CD33 (e.g. gemtuzumab ozogamicin), GM-CSF (e.g. KB002, MT203), GM-CSF receptor (e.g. CAM-3001), EpCAM (e.g. adecatumumab), IFN-gamma (e.g. NI-0501), IFN-alpha (e.g. MEDI-545/MDX-1103), RANKL (e.g. denosumab), hepatocyte growth factor (e.g. AMG 102), IL-15 (e.g. AMG 714), TRAIL (e.g. AMG 655), insulin-like growth factor receptor (e.g. AMG 479, R1507), IL-4 and IL13 (e.g. AMG 317), BAFF/BLyS receptor 3 (BR3) (e.g. CB1), CTLA-4 (e.g. ipilimumab).

In specific embodiments, said antibody is selected from the group consisting of an anti-CD4 antibody (see e.g. WO 97/13852), an anti-CD11a antibody (see e.g. WO 98/23761) and an anti-CD25 antibody (see e.g. WO 2004/045512).

In another embodiment of the invention, the protein according to the invention is an Fc-fusion protein.

In specific embodiments, said Fc-fusion protein comprises a fragment selected from the group consisting of a fragment of TNF (e.g. onercept, etanercept), a fragment of CD28 (e.g. abatacept), a fragment of the TACI receptor, a fragment of the BAFF/BLyS receptor 3 (BR3), an interferon (IFN) or a fragment thereof, and FSH or a fragment thereof.

In one specific embodiment, said Fc-fusion protein comprises a fragment of the TACI receptor (see e.g. WO 02/094852). In another specific embodiment, said Fc-fusion protein comprises IFN-beta (see e.g. WO 2005/001025).

In still another embodiment of the invention, the protein according to the invention is any of the chimeric proteins described in WO 2005/001025. In specific embodiments, such a chimeric polypeptide is selected from the group consisting of:
a) a chimeric protein comprising a first and second polypeptide chain, wherein said first chain comprises a biologically active molecule, and at least a portion of an immunoglobulin constant region and wherein said second chain comprises at least a portion of an immunoglobulin constant region without a biologically active molecule or immunoglobulin variable region;
b) a chimeric protein comprising a first and second polypeptide chain, wherein said first chain comprises a biologically active molecule, and at least a portion of an immunoglobulin constant region and wherein said second chain consists of at least a portion of an immunoglobulin constant region and optionally an affinity tag;
c) a chimeric protein comprising a first and second polypeptide chain, wherein said first chain comprises a biologically active molecule, and at least a portion of an immunoglobulin constant region and wherein said second chain consists essentially of at least a portion of an immunoglobulin constant region and optionally an affinity tag;
d) a chimeric protein comprising a first and second polypeptide chain a) wherein said first chain comprises a biologically active molecule, at least a portion of an immunoglobulin constant region, and a first domain having at least one specific binding partner; and b) wherein said second chain comprises at least a portion of an immunoglobulin without a biologically active molecule or immunoglobulin variable region and further comprising a second domain said second domain being a specific binding partner of said first domain;
e) a chimeric protein comprising a first and second polypeptide chain a) wherein said first chain comprises a biologically active molecule, at least a portion of an immunoglobulin constant region, and a first domain having at least one specific binding partner; and b) wherein said second chain consists of at least a portion of an immunoglobulin, a second domain said second domain being a specific binding partner of said first domain and optionally an affinity tag;
f) a chimeric protein comprising a first and second polypeptide chain a) wherein said first chain comprises a biologically active molecule, at least a portion of an immunoglobulin constant region, and a first domain having at least one specific binding partner; and b) wherein said second chain consists essentially of at least a portion of an immunoglobulin, and a second domain said second domain being a specific binding partner of said first domain and optionally an affinity tag;
g) a chimeric protein of the formula X-La-F:F or F:F-La-X wherein X is a biologically active molecule, L is a linker, F is at least a portion of an immunoglobulin constant region and, a is any integer or zero;
h) a chimeric protein comprising a first and a second polypeptide chain linked together, wherein said first chain comprises a biologically active molecule and at least a portion of an immunoglobulin constant region, and said second chain comprises at least a portion of an immunoglobulin constant region without the biologically active molecule of the first chain and wherein said second chain is not covalently bonded to any molecule having a molecular weight greater than 2 kD;
i) a chimeric protein comprising a first and a second polypeptide chain linked together, wherein said first chain comprises a biologically active molecule and at least a portion of an immunoglobulin constant region, and said second chain comprises at least a portion of an immunoglobulin constant region not covalently linked to any other molecule except the portion of an immunoglobulin of said first polypeptide chain;
j) a chimeric protein comprising a first and a second polypeptide chain linked together, wherein said first chain comprises a biologically active molecule and at least a portion of an immunoglobulin constant region, and said second chain consists of at least a portion of an immunoglobulin constant region; and
k) a chimeric protein comprising a first and a second polypeptide chain linked together, wherein said first chain comprises a biologically active molecule and at least a portion of an immunoglobulin constant region, and said second chain comprises at least a portion of an immunoglobulin constant region without the biologically active molecule of the first chain and a molecule with a molecular weight less than 2 kD covalently attached.

In a specific embodiment, the method according the present invention further comprises the step of calculating the ratio of (i) the amount of first protein relatively to the amount of second protein in a sample; or (ii) the amount of second protein relatively to the amount of first protein in a sample. In another specific embodiment, the method according to the present invention further comprises the step of calculating the percentage of the first or of the second protein relatively to the total amount of said first and second proteins. For example, this can be made using the software of the chromatography system (e.g., the RP-HPLC system) used when performing step (c). Many such software are known in the art and include, e.g., the Empower Software commercialized by Waters.

Another aspect pertains to the use of the method of any of claims 1 to 22 for the validation of manufacturing lots of a therapeutic protein.

Still another aspect pertains to use of a peptide of Formula III for the detection of intact Fc-containing proteins. Said peptide may for example have the sequence of SEQ ID NO: 2, or be a single polymorphic variant thereof.

A further aspect is directed to the use of a peptide of Formula IV for the detection of truncated Fc-containing proteins. Said peptide may for example have the sequence of SEQ ID NO: 3, or be a single polymorphic variant thereof.

All references cited herein, are hereby incorporated by reference in their entirety.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

Having now described the invention, it will be more readily understood by reference to the following examples that are provided by way of illustration and are not intended to be limiting of the present invention.

### EXAMPLES

The following Examples 1 to 3 illustrate the analysis of C-terminal truncation of the TACI-Fc fusion protein described in WO 02/094852 using a method in accordance with the invention. Example 4 compares the results obtained for different antibodies and Fc fusion proteins when using a method in accordance with the invention.

### Example 1

### Hydrolysis of TACI-Fc by Lys-C

5 μg of lyophilized endoproteinase Lys-C (Roche, product No. 1047825) was suspended in 51 μL of purified water.

A sample comprising the TACI-Fc fusion protein was diluted in purified water in order to obtain a concentration of 10 mg/mL. 10 μL (100 μg) of this dilution was added to 100 μL of a buffer solution at pH 7.5 comprising 0.5M Tris-HCl and 2 mM EDTA. 25 μL of Lys-C endoproteinase were then added. The proteolytic mixture was slowly mixed using a vortex and incubated for 2 hours±10 min at 37±2° C. The reaction was the stopped by adding 10 μL of trifluoroacetic acid (TFA, J.T. Baker, product No. 9470) at a concentration of 10% to the proteolytic mixture.

The control (blank) was prepared by adding 25 μL of Lys-C endoproteinase to 110 μL of the buffer solution at pH 7.5 comprising 0.5M Tris-HCl and 2 mM EDTA. The control was incubated and the reaction stopped as described above.

### Example 2

### Analysis of the Proteolytic Mixture by RP-HPLC Chromatography

### 2.1. Material

The RP-HPLC was performed using an “Alliance” HPLC (Waters) equipped with oven for the column. The HPLC was equipped with two columns:
an analytical column C18, 5 μm (4.6 mm×50 mm) (Vydac, Product No. 218TP5405); and
an HP Guard column C18, 5 μm (Vydac, Product No. 218GD54).

### 2.2. Conditions

The HPLC lines were connected with the following solutions:
Line A: 0.10% Trifluoroacetic acid in water (referred to as eluent A)
Line B: 0.08% Trifluoroacetic acid in Acetonitrile 70% (referred to as eluent B)
   The analytical column and the guard column were connected to the instruments and the following parameters were inputted:
UV Detection: 214 nm
Column temperature: +40° C.
Autosampler temperature: +5° C.
Autosampler Loop: 2 mL
Syringe: 250 μL
Column flow rate: 1 mL/min
Run duration: 15 min
   The linear gradient was set up as indicated in table 4.

TABLE 4

| Time | Flow | | | |
|---|---|---|---|---|
| (min) | (mL/min) | % A | % B | Curve |
| 0 | 1.00 | 85 | 15 | — |
| 5.00 | 1.00 | 85 | 15 | 1 |
| 6.00 | 1.00 | 79 | 21 | 6 |
| 7.00 | 1.00 | 77 | 23 | 6 |
| 7.10 | 1.00 | 0 | 100 | 6 |
| 10.00 | 1.00 | 0 | 100 | 6 |
| 10.10 | 1.00 | 85 | 15 | 1 |
| 15.00 | 1.00 | 85 | 15 | 1 |

The column was first equilibrated by flushing the column with the mobile phase in the starting composition (85% eluent A, 15% eluent B) at 1 mL/min for not less than 10 minutes, keeping the column at 40° C. The equilibration was completed when the baseline was stable. 60 to 75 μL of the proteolytic mixture obtained in Example 1 were injected into the column for each analysis.

### 2.3. Results

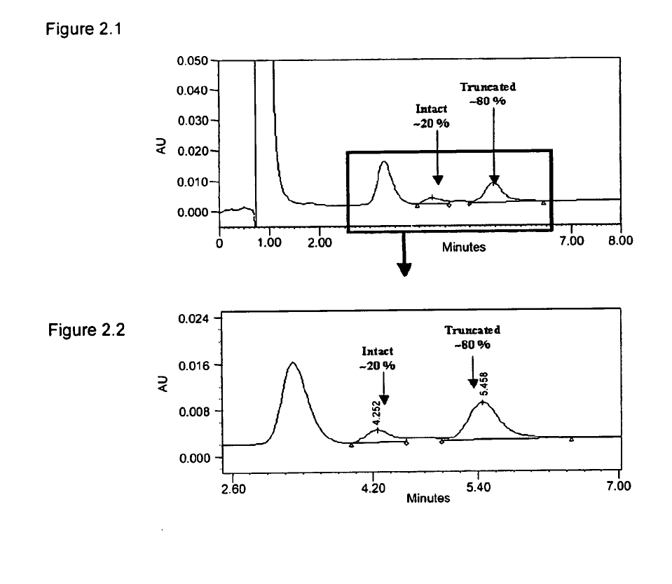

A typical RP-HPLC analytical profile obtained for TACI-Fc is shown in FIG. 2.

### Example 3

### Quantification of the C-Terminal Truncation of TACI-Fc

The analysis of Example 2 was performed twice for each proteolytic mixture obtained in Example 1 and the peaks detected as shown in FIG. 2 were integrated, i.e., the peaks of interest were identified and their area was determined by tracing the baseline, either manually or automatically. The percentage of TACI-Fc truncated at its C-terminus was directly calculated by the software of the RP-HPLC system (Empower Software, Waters). The percentage of truncated or intact molecules corresponds to the value referred to as “area %”. The final percentage of TACI-Fc truncated at its C-terminus was obtained by calculating the average percentage between the two replicates.

In FIG. 2, the TACI-Fc sample comprises about 20% Lys1 variants and about 80% Lys0 variants.

### Example 4

### Analysis of C-Terminal Truncation of Six Different Proteins

The six following proteins comprising the sequence of SEQ ID NO: 1 at their C-terminal extremity were analyzed using the protocol described in Examples 1 to 3:
the TACI-Fc fusion protein analyzed in Examples 1 to 3;
the anti-CD4 antibody 6G5 described in WO 97/13852 (Anti-CD4 Mab);
the anti-CD25 antibody AB12 described in WO 2004/045512 (Anti-CD25 Mab)
the Fc-fusion protein comprising IFN-beta described in WO 2005/001025 (IFN-Fc No. 1)
an alternative Fc-fusion protein comprising a fragment of IFN-beta (IFN-Fc No. 2); and
the anti-CD11a antibody F(ab)-8 described WO 98/23761 (Anti-CD11a Mab).

The results are shown in FIG. 3. The percentage of C-terminal truncation could be calculated for all six proteins without changing the experimental conditions set forth in Examples 1 to 3. Thus the method in accordance to the invention is a widely applicable method for analyzing C-terminus truncation of monoclonal antibodies and of Fc-fusion proteins.

### REFERENCES

1. Dillon, T. M., Bondarenko, P. V., and Speed, R. M. (2004) Development of an analytical reversed-phase high-performance liquid chromatography-electrospray ionization mass spectrometry method for characterization of recombinant antibodies. J. Chromatogr. A 1053, 299-305.
2. Lazar, A. C., Kloczewiak, M. A., and Mazsaroff, I. (2004) Matrix-assisted laser desorption/ionization mass spectrometry for the evaluation of the C-terminal lysine distribution of a recombinant monoclonal antibody. Rapid Commun. Mass Spectrom. 18, 239-244.
3. Santora, L. C., Krull, I. S., and Grant, K. (1999) Characterization of recombinant human monoclonal tissue necrosis factor-alpha antibody using cation-exchange HPLC and capillary isoelectric focusing. Anal. Biochem. 275, 98-108.

1-23. (canceled)

24. A method measuring the relative amount of a first protein and of a second protein in a sample, said method comprising the steps of:
(a) providing a sample comprising said proteins;
(b) hydrolyzing said proteins with a Lys-C endoproteinase; and
(c) separating the hydrolysate obtained in step (b) by a method capable of distinguishing between peptides having a difference of one amino acid in length;
wherein:
(i) said first protein comprises a peptide of Formula I at its C-terminal extremity:
Lys-(Xaa)z-Lys Formula I
(ii) said second protein comprises a peptide of Formula II at its C-terminal extremity:
Lys-(Xaa)z Formula II
(iii) Xaa is any amino acid except of Lys;
(iv) 5≦z≦20; and
(v) the sequence of said first protein is identical to the sequence of said second protein except for the additional presence of a C-terminal lysine in said first protein.

25. The method of claim 24, wherein the method of step (c) distinguishes between peptides of Formula III and Formula IV:
(Xaa)z-Lys Formula III
(Xaa)z. Formula IV

26. The method of claim 24, wherein said first protein is an Fc-containing protein.

27. The method of claim 24, wherein said first protein comprises the sequence of SEQ ID NO: 1 at its C-terminal extremity.

28. The method of claim 24, wherein said first protein sequence comprises a single polymorphic variant of SEQ ID NO: 1 at its C-terminal extremity.

29. The method of claim 24, wherein the method of step (c) distinguishes between peptides having a sequence of SEQ ID NO: 2 and peptides having a sequence of SEQ ID NO: 3.

30. The method of claim 24, wherein step (c) is carried out by chromatography.

31. The method of claim 30, wherein step (c) is carried out by Reverse Phase High Performance Liquid Chromatography (RP-HPLC).

32. The method of claim 30, wherein the temperature of the chromatography column is of about 40° C.

33. The method of claim 30, wherein said RP-HPLC is performed using:
(i) 0.10% trifluoroacetic acid in water; and
(ii) 0.08% trifluoroacetic acid in acetonitrile 70%.

34. The method of claim 24, wherein step (b) is carried out with 5 μg of said Lys-C endoproteinase and with 100 μg of said protein.

35. The method of claim 24, wherein step (b) is carried out for about 2 hours.

36. The method of claim 24, wherein step (b) is carried out at about 37° C.

37. The method of claim 24, further comprising the step of stopping the reaction of step (b) before carrying out step (c).

38. The method of claim 24, wherein said sample comprises purified proteins.

39. The method of claim 24, wherein said sample is a pharmaceutical preparation.

40. The method of claim 24, wherein said first protein is an antibody.

41. The method of claim 40, wherein said antibody is a monoclonal antibody.

42. The method of claim 41, wherein said monoclonal antibody is an antibody selected from the group consisting of a chimeric antibody, a humanized antibody and a human antibody.

43. The method of claim 40, wherein said antibody is selected from the group consisting of an anti-CD4 antibody, an anti-CD 11a antibody and an anti-CD25 antibody.

44. The method of claim 24, wherein said first protein is an Fc-fusion protein.

45. The method of claim 44, wherein said Fc-fusion protein comprises either a fragment of the TACI receptor or IFN-beta.


SEQUENCE LISTING (4 sequences)

SEQ ID NO: 1 - protein, 9 aa, Artificial
  Nine C-terminal amino acids of heavy chains of antibodies and of Fc chains
KSLSLSPGK

SEQ ID NO: 2 - protein, 8 aa, Artificial
  Intact sequence present in heavy chains of antibodies and in Fc chains
SLSLSPGK

SEQ ID NO: 3 - protein, 7 aa, Artificial
  Truncated sequence present in heavy chains of antibodies and in Fc chains
SLSLSPG

SEQ ID NO: 4 - protein, 157 aa, Artificial
  Fc chain comprising SEQ ID NO 1 at its C-terminal extremity
PREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK